(19) United States
(12) Patent Application Publication
(10) Pub. No.: US 2012/0283637 A1
(43) Pub. Date: Nov. 8, 2012

(54) Injection site marker

(76) Inventor: Cynthia E. Cohen, El Paso, TX (US)

(21) Appl. No.: 12/925,554

(22) Filed: Oct. 25, 2010

Related U.S. Application Data
(60) Provisional application No. 61/280,339, filed on Nov. 3, 2009.

(51) Int. Cl. A61M 5/42

(52) U.S. Cl. 604/116

(57) ABSTRACT

The present invention relates to the use of syringes for medicinal liquid injections into mammals, particularly humans, with the syringe bearing a marker ink to mark injection site with marker ink. The disclosed embodiments address re-injecting a medical liquid into the skin at the same location as previous injections. The practice of re-injecting medical liquids into the same location can result in indentions, hard lumps, or thickening of the skin, or lipohyperplasia. It can further cause medications such as insulin, to be inefficiently absorbed. Rotating injection sites within arms, thighs, stomach or buttock usually prevents or reduces this problem. A marker is used on the portion of the syringe that touches the skin of a recipient and causes ink or powder to be transferred to the skin of the recipient to mark an injection site for a desired length of time.

### RELATED CASES

The present invention is entitled to and hereby claims the benefit of the filing date of U.S. Provisional Application Ser. No. 61/280,339 filed Nov. 3, 2009 by Cindy Cohen entitled “Injection Site Marker.”

### FIELD OF THE INVENTION

The present invention relates to the use of syringes for injections into mammals, particularly humans.

### BACKGROUND OF THE INVENTION

This invention pertains to the medical field and solves the problem of re-injecting a medicinal liquid into the skin at the same location where previous injections have been made. The practice of re-injecting medicinal liquids into the same location can result in indentations, hard lumps or thickening of the skin and can cause medications, such as insulin, to not be absorbed efficiently. Rotation of injection sites within arms, thighs, stomach or buttock usually prevents or reduces this problem.

This invention also assists in the identification of used syringes. Spread of disease, i.e., HIV and hepatitis, by reuse of syringes has been demonstrated in numerous instances. Most recently, in 2008, in Las Vega, Nev., an endoscopy center reused syringes on different patients which resulted in the spread of infection.

This invention uses a marker on the portion of the syringe which touches the skin of a recipient and causes ink or powder to be transferred to the skin of the recipient. Contact with the skin would transfer the marking material to the recipient and would render the syringe unable to remark skin. If a patient sees that the end of the syringe does not exhibit any powder or ink, the recipient would be alerted that the syringe had possibly been previously used, with the aim that all syringes in clinical use would be safeguarded for a single use only.

Accordingly, a continuing effort has been directed to the development of a method for marking the site of injections so that the marking is visible for a period of time and is removable after a desired period of time (typically 24 to 48 hours). Desirably a marking ink is positioned on the syringe so that the powder or ink does not contact the injection needle during injection to help ensure needle sterility.

### SUMMARY OF THE INVENTION

According to the present invention, a method for marking an injection site of a dose of a medicinal liquid injection into a recipient from a syringe, comprising: a chamber adapted to contain the medicinal liquid, a plunger adapted to move the medicinal liquid out of the chamber through a needle in fluid communication with the chamber, with a shoulder on the syringe positioned around the needle orthogonal to a longitudinal axis of the syringe and insertable into the recipient, the method consisting essentially of; positioning a marking ink on the shoulder of the syringe; inserting the needle into the recipient to a depth sufficient to move the shoulder of the syringe into marking contact with the recipient; injecting the dose of medicinal liquid into the recipient; and, withdrawing the needle from the recipient.

The invention further includes a marking syringe for injection of a medicinal liquid into a mammal, the syringe comprising a chamber adapted to contain the medicinal liquid, a plunger adapted to move the medicinal liquid out of the chamber though a needle having a base in an end of the chamber, in fluid communication with the chamber and insertable into the mammal, a cap over the needle and in engagement at its first end with an outside of the syringe, the improvement consisting of a shoulder positioned at a base of the needle, orthogonal to a longitudinal axis of the syringe and adapted to contact the skin of the mammal when the needle is used for injection of the medicinal liquid and a marker ink positioned on the shoulder to mark an injection site on the skin of the mammal when the syringe is used.

In an alternate embodiment of the present invention further a method is provided for marking an injection site of a dose of a medicinal liquid injection into a recipient from a syringe, comprising: a chamber adapted to contain the medicinal liquid, a plunger adapted to move the medicinal liquid out of the chamber through a needle in fluid communication with the chamber, with a shoulder on the syringe positioned around the needle and insertable into the mammal, a cap over the needle and in engagement at its first and with an outside of the chamber and having an end zone positioned on the syringe and orthogonal to an axis of the syringe, the method consisting essentially of; positioning a marking ink on the end zone; removing the cap from engagement with the outside of the syringe; pressing the end zone of the cap against a target area on the recipient for injection of the dose of medicinal liquid; inserting the needle into the recipient in the target zone; injecting the dose of medicinal liquid and withdrawing the needle.

An alternate embodiment of the invention provides a syringe for injection of a medicinal liquid into a recipient, the syringe comprising a chamber adapted to contain the medicinal liquid, a plunger adapted to move the medicinal liquid out of the chamber though a needle having a base in an end of the chamber, in fluid communication with the chamber and insertable into the recipient, a cap over the needle and in engagement at its first end with an outside of the syringe, the improvement consisting of an end zone positioned on the first end of the cap orthogonal to a longitudinal axis of the syringe and the cap, a marker ink positioned on the end zone so that the cap can be removed from the outside of the syringe so the cap end zone can be pressed against the recipient to mark an injection location.

### BRIEF DESCRIPTION OF THE DRAWINGS

FIG. 1 is a schematic drawing of a syringe for injecting a medicinal liquid into a mammal;

FIG. 2 is a cross-sectional view taken at lines aa of FIG. 1;

FIG. 3 is cross-sectional view of a modified end portion of a syringe as shown in FIG. 1;

FIG. 4 is a view of a portion of an abdominal section of a human showing markings positioned by the embodiment shown in FIG. 1 and by the embodiment of FIG. 3′

FIG. 5 is a schematic view of a pen needle;

FIG. 6 is a cross-sectional view of the chamber of the pen needle; and,

FIG. 7 is an embodiment for a cover for a pen needle.

### DESCRIPTION OF PREFERRED EMBODIMENTS

In the discussion of the Figures, the same numbers will be used throughout to refer to the same or similar components.

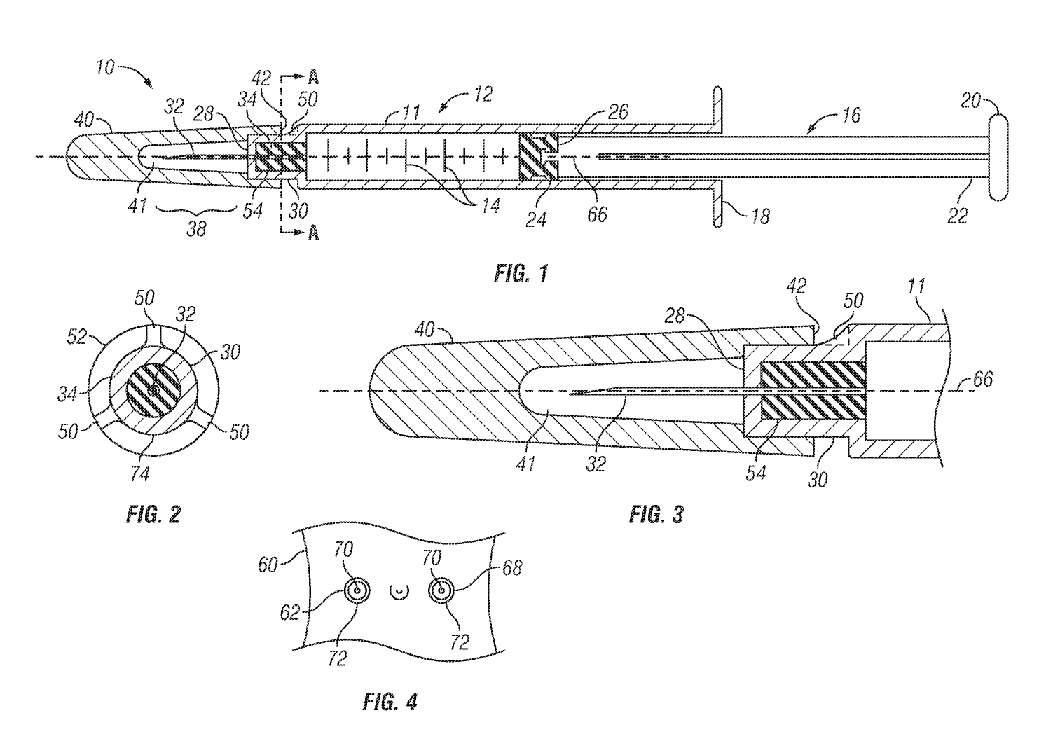

In FIG. 1 a syringe 10, including a chamber 12, having an outside 11 and markings 14 is shown. A plunger 16 is shown positioned in a first end 18 of chamber 12 and having a handle 20. First end 18 is configured to allow engagement by fingers with the thumb being positioned on handle 20 to push plunger 16 toward a second end 28 of the chamber. A second end 26 of plunger 16 includes seals 24 which are positioned to push a liquid contained in chamber 12 outwardly through a passageway 34 in the second end of the chamber. The chamber includes markings 14 which enable the user to determine the size of a medicinal dose ejected from the chamber. The material ejected from the chamber is passed through a needle 32 which in use is inserted into a recipient of a dose of medicinal liquid.

As typically provided, syringe 10 includes a cap 40 which is positioned over a section 30 of chamber 12 to protect needle 32. Cap 40 also includes a shoulder 36 which engages an end portion of section 30 which is a reduced diameter end portion of chamber 12. Needle 32 is positioned in a cavity 41 in cap 40 as shown. In use cap 40 is removed from needle 32, which is desirably of a length compatible with the depth of the injection into the recipient. When needle 32 is inserted into the recipient, an end portion section 30 of chamber 12 (section 30 may be of different sizes and could be the same diameter as chamber 12) which has been in contact with shoulder 36 on cap 40 comes into contact with the flesh of the recipient.

According to the present invention, a marking is placed on the end portion of section 30 so that when section 30 engages the skin of the recipient, the ink marking (or a marking powder which is included in the term marking ink) is deposited on the skin of the recipient at the site of the injection. The ink may be placed on the end portion of reduced diameter section 30 by any suitable means.

As shown in FIG. 1, the ink could be placed on the reduced diameter section 30 by positioning ink on shoulder 36 of cap 40 so that the ink is transferred to the outer portion of reduced diameter section 30. Alternately the ink could be placed on the entire end surface of the reduced diameter section 30, although it is preferred that the ink be placed only on the outer circumferential portion of the reduced diameter section 30.

The marker ink forms a mark on the skin of the user as shown for instance, in FIG. 4 (which depicts the torso of a recipient), by mark 62 wherein the ink typically forms a pattern such as shown as mark 62 when ink is placed around the entire end surface of reduced diameter section 30 so that the mark is a round mark surrounding the needle puncture mark 70. In an alternate embodiment wherein the ink is deposited only on the outer portion of reduced diameter section 30, a mark is shown at mark 68 is produced wherein the ink surface mark 72 surrounds the needle puncture mark 70. In either case, the ink 70 which is deposited on the skin of the recipient is desirably an ink which is readily removed by contact and which is durable enough to provide a mark for a desired length of time. Typically the ink is readily removed at the expiration of the desired length of time.

A wide variety of appropriate inks is available for marking the skin to provide a mark of varying durability. One such ink is marketed for marking on the palm of a hand by “fredandfriends”, Cumberland, R.I. The product is marketed as a pen for positioning markings on the palm of a user to stimulate memory. The ink is marketed in a marker under the trademark “TO-DO TATTOO”.

The end portion of reduced diameter section 30 may have a configuration at line A-A as shown in FIG. 2. An outer circumference 52 of chamber 12 is shown. The external outer circumference 74 of reduced diameter section 30 is shown with projections 50 positioned thereon to engage an inner surface 54 of cap 40. This configuration is useful when a first end 42 of cap 40 is coated with ink.

When end 42 of cap 40 is coated with ink, the marking is produced by removing the cap, pressing it against the flesh of the recipient at the desired injection site and then injecting the needle in the marked area. It will be noted that in both embodiments of the invention, the needle is injected into the flesh of the recipient or at an area which is not marked at the injection point before any ink is positioned onto the skin of the recipient. Accordingly, the recipient is protected against the injection of any marking ink used to produce the marking.

In FIG. 3, an alternate embodiment of cap 40 is shown where no cap shoulder is placed in contact with the shoulder 36 formed by the end of reduced diameter section 30. In this embodiment a shoulder (end zone) is formed on first end 42 of cap 40. Ink is placed on this shoulder, and as indicated previously, the cap is removed for marking the skin of the recipient at a desired location.

Syringe 10 includes a central axis 66 and is typically symmetrically positioned around axis 66.

Having described the syringe, it is pointed out that either the marking ink may be positioned on a shoulder 36 on an end of reduced diameter section 30 for positioning on the skin of the recipient when the needle is inserted full length into the recipient. Alternatively, the mark may be positioned on the torso of the recipient by placing ink on a first end 42 of cap 40 for removal and contacting with the skin at a desired injection site on the recipient. The needle may then be inserted into the center of the marked area and the medicinal liquid is injected.

In the event that it is desirable to prevent the presence of ink in the vicinity of the injection site, even after the injection has been completed, then the embodiment described in FIG. 3 is preferred. In this embodiment, the ink is in all cases absent from the injection area while still marking the injection area.

While the torso of a human is shown as the injection area in FIG. 4, the injection could be at any part of the body of a recipient of the medicinal liquid. As indicated previously, the medicinal liquid may be injected at reasonably frequent intervals and especially if injected by the recipient. Medicinal liquids which are frequently injected into the torso of a recipient include insulin and other drugs requiring frequent injection doses

The method and syringe of the present invention are useful with any portion of the body and may be used with injections which are not frequent but which are otherwise desirably readily located after the injection is complete.

While a simple syringe has been shown, it should be recognized that automated syringes may be used or larger capacity syringes may be used and the like.

The length or capacity of the syringe is not critical to the practice of the present invention, provided that the ink surfaces as described are available and are readily placed in contact with the skin of the recipient.

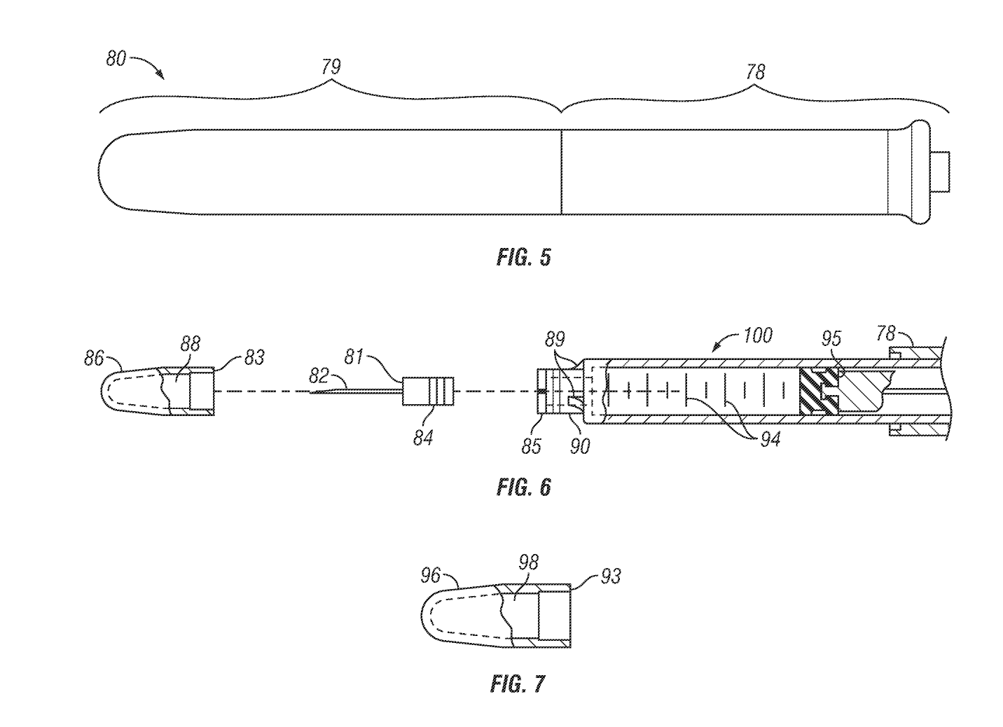

An alternate embodiment with a pen needle is shown in FIG. 5 through FIG. 7. In FIG. 5 a pen needle is shown and comprises a first section 78 which enables the user to select the size of dosage for a particular injection and to make the injection. The pen contains more medicinal liquid than required for each injection and thus can be used for numerous injections.

A cover 30 covers the cylinder and needle and associated components. The cylinder and needle are shown in more detail in FIG. 6. A new needle is used for the first and for each subsequent injection using the pen needle. The chamber is operated substantially as discussed above and contains markings 14 and the like which are indicative of the amount of medicinal liquid selected for injection and also indicates the amount of medicinal liquid remaining in the pen needle. As indicated, the amount of medicinal liquid injected can be dialed into the pen needle prior to injection. A reduced diameter section 30 of chamber 12 is shown and includes threads 82. The needle is provided with a threaded fitting at one of its ends so that it is readily screwed onto reduced diameter section 30.

The sleeve 86 over the needle is then removed and the needle is ready for use. As delivered to the user, the pen needle typically also includes an outer cap 90. This cap is positioned over the sleeve and needle. It typically engages an outside of reduced diameter section 30 of chamber 12. This cap includes a cavity 92 which is sized to go over needle 36, base 84 and sleeve 86. This sleeve protects these components from contact with foreign materials and the like.

In the practice of the present invention, a surface 36 on base 84 is coated with a marking ink which is transferred to the recipient of the needle when the needle is inserted for injection.

The ink may be positioned on the needle by any desired means. The marking ink may be positioned on the ends of caps by contacting the ends with a marking ink source such as a marking ink pad, roller or the like. Marking ink is readily positioned on the cap contacting the shoulder of the syringe.

In an alternate embodiment, cap 90 may be equipped with a marking ink on its first end 42 so that when it is removed it can be used by placing it in position on a recipient to transfer ink to the recipient to mark the site of the injection.

As discussed above, this particular technique does not result in ink being positioned immediately on the puncture mark but rather around the puncture mark.

While either of the embodiments disclosed can be used, as well as a number of other embodiments, it is important to the present invention that a marking ink be positioned on a base of the needle so that the location is marked so that the user of the needle can tell where injections have previously been made and the like. Similarly, the use of the cap to mark the site is equally effective, and as indicated above, does not result in ink be positioned immediately adjacent the needle injection point.

While the present invention has been described by reference to certain of its preferred embodiments, it is pointed out that the embodiments described are illustrative rather than limiting in nature and that many variations and modifications are possible within the scope of the present invention. Many such variations and modifications may be considered obvious and desirable by those skilled in the art based upon a review of the foregoing description of preferred embodiments.

1-12. (canceled)

13. A method comprising the steps of:
visibly marking an injection site for an injection from an injector; and
retaining said marked injection site for a length of time to indicate rotation of any subsequent injection from said marked injection site to prevent re-injecting said subsequent injection into said injection site.

14. The method of claim 13 further comprising:
inserting a needle of said injector into a recipient at a sufficient depth to move a shoulder of said injector into marking contact with said recipient.

15. The method of claim 13 further comprising:
capping a needle of said injector with a cap by engaging a first end of said cap with an exterior portion of said injector.

16. The method of claim 15 wherein said cap comprises a shoulder positioned around a needle of said injector, orthogonal to a longitudinal axis of said injector, and in contact with a shoulder of said injector.

17. The method of claim 15 wherein said cap comprises a shoulder covered with a marking ink to mark around or proximate to said injection site when an end portion section of a chamber of said injector contacts said marking ink on said cap shoulder to avoid contact with a needle of said injector.

18. The method of claim 17 wherein said marking ink comprises at least one of a durable marking ink, marking powder, durable marking ink that is readily removed from said injection site after a period of time, and durable marking powder that is readily removed from said injection site after a period of time.

19. The method of claim 13 wherein said injection comprises a medicinal liquid dose intended for injection into a recipient.

20. The method of claim 13 wherein said injector comprises at least one of the following: a syringe and a pen needle injector.

21. A method comprising the steps of:
contacting marking ink on a cap of an injector with an injection site;
visibly marking said injection site for an injection from said injector; and
retaining said marked injection site for a length of time to indicate rotation of any subsequent injection from said marked injection site to prevent re-injecting said subsequent injection into said injection site.

22. The method of claim 21 further comprising injecting said injection from said injector around or proximate to said visible marking from said marking ink contacting said injection site wherein said injector comprises at least one of the following: a syringe and a pen needle injector.

23. The method of claim 21 wherein said marking ink comprises at least one of a durable marking ink, marking powder, durable marking ink that is readily removed from said injection site after a period of time, and durable marking powder that is readily removed from said injection site after a period of time.

24. A system comprising:
an injector; and
a visibly-marked injection site where an injection from said injector is received wherein said marked injection site is retained for a length of time to indicate rotation of any subsequent injection from said marked injection site to prevent re-injecting said subsequent injection into said injection site.

25. The system of claim 24 further comprising:
a needle of said injector that is inserted into a recipient at a sufficient depth to move a shoulder of said injector into marking contact with said recipient wherein said injector comprises at least one of the following: a syringe and a pen needle injector.

26. The system of claim 24 further comprising:
a cap for capping a needle of said injector by engaging a first end of said cap with an exterior portion of said injector.

27. The system of claim 26 wherein said cap comprises a shoulder positioned around said needle of said injector, orthogonal to a longitudinal axis of said injector, and in contact with said shoulder of said injector.

28. The system of claim 26 wherein said cap comprises a shoulder covered with a marking ink to mark around or proximate to said injection site when an end portion section of a chamber of said injector contacts said marking ink on said cap shoulder to avoid contact with said needle of said injector.

29. The system of claim 28 wherein said marking ink comprises at least one of a durable marking ink, marking powder, durable marking ink that is readily removed from said injection site after a period of time, and durable marking powder that is readily removed from said injection site after a period of time.

30. An apparatus comprising:
an injector for visibly-marking injection site where an injection from said injector is received wherein said marked injection site is retained for a length of time to indicate rotation of any subsequent injection from said marked injection site to prevent re-injecting said subsequent injection into said injection site.

31. The apparatus of claim 30 further comprising:
a cap for capping a needle of said injector by engaging a first end of said cap with an exterior portion of said injector, wherein said cap comprises a shoulder positioned around said needle of said injector, orthogonal to a longitudinal axis of said injector, and in contact with said shoulder of said injector, wherein said cap shoulder is covered with a marking ink to mark around or proximate to said injection site when an end portion section of a chamber of said injector contacts said marking ink on said cap shoulder to avoid contact with said needle of said injector wherein said injector comprises at least one of the following: a syringe and a pen needle injector.

32. The apparatus of claim 31 wherein said marking ink comprises at least one of a durable marking ink, marking powder, durable marking ink that is readily removed from said injection site after a period of time, and durable marking powder that is readily removed from said injection site after a period of time.
